(19) United States
(12) Patent Application Publication
(10) Pub. No.: US 2012/0115171 A1
(43) Pub. Date: May 10, 2012

(54) Method For Diagnosing Thrombophilia

(75) Inventors: Sebastien Lacroix-des-Mazes, Paris (FR); Vincent Mallet, Paris (FR); Srinivas Kaveri, Paris (FR); Stanislas Pol, Paris (FR)

(73) Assignees: INSERM (INSTITUT NATIONAL DE LA SANTE ET DE LA RECHERCHE MEDICALE), Paris (FR); ASSISTANCE PUBLIQUE HOPITAUX DE PARIS, Paris (FR); UNIVERSITE RENE DESCARTES PARIS 5, Paris (FR); CENTRE NATIONAL DE LA RECHERCHE SCIENTIFIQUE-CNRS, Paris (FR)

(21) Appl. No.: 13/318,685

(22) PCT Filed: May 7, 2010
(86) PCT No.: PCT/EP2010/056268

§ 371 Date: Jan. 19, 2012

(30) Foreign Application Priority Data
May 7, 2009 (EP) 09305412.0

(51) Int. Cl. G01N 33/566

(52) U.S. Cl. 435/7.92

(57) ABSTRACT

The invention relates to a method for diagnosing thrombophilia in a subject suffering from HIV or from a systemic auto-immune disease, said method comprising determining in a blood sample obtained from said subject the level of free Protein S having Activated Protein C (APC) cofactor activity and the level of total Protein S.

### FIELD OF THE INVENTION

The invention relates to a method for diagnosing thrombophilia.

### BACKGROUND OF THE INVENTION

Goodwin et al (Goodwin et al. Archives of Pathology and Laboratory Medicine: Vol. 126, No. 11, pp. 1349-1366) have examined the technical and diagnostic aspects of protein S (PS) assays and their application to clinical epidemiologic studies. Protein S (PS) is a vitamin K-dependent plasma glycoprotein composed of 635 amino acids that functions as a cofactor in the protein C anticoagulant system. Protein S is produced mainly in the liver.

The association of hereditary PS deficiency and venous thrombosis was first identified in 1984. There have been rare reported cases of arterial thrombosis associated with hereditary PS deficiency. Protein S acts as a cofactor that enhances the activity of activated protein C (APC) in the proteolytic degradation of factors Va and VIIIa.

In plasma, 60% to 70% of PS is noncovalently bound to C4 binding protein (C4bBP, a complement regulatory protein) through a binding site in the carboxy-terminal, steroid-binding globulin domain of PS, with the dissociation constant for the binding interaction in the nanomolar range. This binding affinity predicts that all available C4bBP binding sites for PS will be filled. This binding interaction complicates the measurement and interpretation of PS concentrations in plasma. C4bBP is a multimeric protein with 2 isoforms (Mr 540000 and 590000). The anticoagulant activity of PS resides with free PS. The PS bound to C4bBPβ+ does not have APC cofactor activity.

Protein S deficiency can be hereditary or acquired. Rare cases of homozygous PS deficiency have been associated with severe neonatal purpura fulminans, similar to homozygous protein C deficiency. Also, similar to protein C deficiency, biochemical evidence for PS deficiency suggests a prevalence of about 1 in 500.

The International Society for Thrombosis and Haemostasis Standardization Subcommittee has defined 3 types of hereditary PS deficiency based on the plasma concentration of total PS, free PS, and APC cofactor activity. Type I PS deficiency is identified by low levels of free and total PS antigen, with decreased APC cofactor activity. Type II PS deficiency is characterized by normal levels of total and free PS antigen with low levels of APC cofactor activity. Type III PS deficiency is characterized by normal to low levels of total PS, low free PS, and an elevated fraction of PS bound to C4bBP. Approximately two thirds of PS-deficient patients have type I deficiency, one third have type III deficiency, and type II deficiency is rare.

Plasma PS has been quantitated by immunologic and functional assays. A review by Faioni (Faioni. Thromb Haemost 2001; 86:1139-1140) underlines the long-standing methodologic problems that continue to create uncertainty in the measurement of this complex analyte. Early assays involved measuring total PS by polyclonal electroimmunoassay (primarily Laurell). Free PS was determined by either 2-dimensional immunoelectrophoresis or measuring the supernatant following precipitation of C4bBP-PS complexes in plasma by the addition of 3.75% polyethylene glycol 6000 (PEG). Clinical laboratories now have a number of commercially available polyclonal enzyme-linked immunosorbent assays (ELISAs) that, together with PEG precipitation, measure total and free PS. Although PEG precipitation is notoriously irreproducible and time-consuming, it remains the gold standard for the validation of functional PS assays and monoclonal free PS ELISAs. In recent years, a few commercially available monoclonal ELISAs have become available that accurately measure free PS. The only drawback of the free PS assays is that they might miss rare cases of type II PS deficiency. In 1996, a joint International Society of Thrombosis and Haemostasis and World Health Organization (WHO) meeting recommended that measurement of free PS may be more useful than total PS for the diagnosis of PS deficiency. Furthermore, this consensus conference in 1996 favoured using immunoassays for free PS because of the lack of specificity (primarily due to APC resistance) of the available functional assays.

The APC cofactor activity of PS can be measured in assays based on modified activated partial thromboplastin time (APTT) and prothrombin time (PT) formats. In the APTT format, diluted patient plasma is added to PS-depleted plasma in the presence of purified APC and factor Va. In the PT format, a similar approach can be taken or the native plasma PC in the depleted plasma can be activated by Protac, an enzyme from the Southern Copperhead snake venom (Agkistrodon contortrix contortrix). The drawbacks of the functional assays result from the high rate of false-positive results due to the presence of APC resistance, factor V Leiden, high concentrations of prothrombin, factor VIIIa, and factor VIIa. The lupus-like inhibitor may also interfere, although this effect is minimized by the dilution of patient plasma used in the assay. Assays that use added high concentrations of added factor Va are less affected by the presence of APC resistance or factor V Leiden.

The lower limit of the normal range for plasma total PS concentration is commonly accepted as 65% of the concentration observed in pooled normal human plasma. However, individual laboratories should establish normal ranges specific for their assay using plasma samples obtained from normal individuals. The PS plasma concentrations are lower in women than men, premenopausal compared with postmenopausal women, pregnant women, and women taking oral contraceptions, thus pointing to a significant effect of hormonal status. In general, PS levels increase with age in women, with little change in men.

It is an object of the present invention to provide a simple and reliable method for assessing thrombophilia.

### SUMMARY OF THE INVENTION

The invention relates to a method for diagnosing thrombophilia in a subject suffering from HIV or from a systemic auto-immune disease, said method comprising determining in a blood sample obtained from said subject the level of free Protein S having Activated Protein C (APC) cofactor activity and the level of total Protein S.

Measuring both the level of free Protein S having APC cofactor activity and the level of total Protein S in a blood sample allows to diagnose thrombophilia induced by autoantibodies which inhibit the APC cofactor activity of the Protein S.

By comparing the level of free Protein S having APC cofactor activity with the level of total Protein S, for example by calculating the ratio free Protein S having APC cofactor activity/total Protein S, one can obtain a normalized value which is substantially independent of Protein S variations due for example to vitamin K deficiency, liver diseases, pregnancy, age . . . . This normalized value then provides a reliable indicator of autoimmune protein S deficiency.

Typically, the ratio free Protein S having APC cofactor activity/total Protein S is an indirect marker for the presence of anti-protein S autoantibodies that neutralize the cofactor activity of the free protein S. A low ratio is indicative of the presence of anti-protein S autoantibodies that neutralize the cofactor activity of the free protein S. The invention also relates to the use of free Protein S having APC cofactor activity and of total Protein S as indicators of autoimmune protein S deficiency.

Typically this ratio can be compared with a reference value. The reference value may be obtained from a group of healthy subjects. Typically the subjects may have similar sex, age, and/or body mass index as compared with the subject from which the biological sample to be tested was obtained. Alternatively the reference value may be obtained from a group of subjects suffering from HIV with nodular regenerative hyperplasia or from a systemic auto-immune disease.

The invention also relates to the use of free Protein S having APC cofactor activity and of total Protein S as indicators of autoimmune protein S deficiency. The invention also relates to a method for diagnosing autoimmune protein S deficiency in a subject, said method comprising determining in a blood sample obtained from said subject the level of free Protein S having Activated Protein C (APC) cofactor activity and the level of total Protein S.

Another object of the invention relates to a kit comprising:

a) means for detecting free Protein S having APC cofactor activity; and
b) means for detecting total Protein S.

### DETAILED DESCRIPTION OF THE INVENTION

The term “thrombophilia” refers to an abnormality of haemostasis predisposing to thrombosis.

The term “blood sample” as used herein refers to a blood sample obtained for the purpose of in vitro evaluation. Examples of blood samples are a whole blood sample, a plasma or a serum sample.

The invention relates to a method for diagnosing thrombophilia in a subject suffering from HIV or from a systemic auto-immune disease, said method comprising determining in a blood sample obtained from said subject the level of free Protein S having Activated Protein C (APC) cofactor activity and the level of total Protein S.

In an embodiment, the systemic auto-immune disease is systemic lupus erythematosus (SLE), the antiphospholip syndrome, Behcet's disease, Common variable immune deficiency (CVID), viral-induced prothrombotic states, thrombotic thrombocytopenic purpura.

It has already been shown that acquired APC resistance is associated with Anti-protein S autoantibodies in patients with SLE (Nojima et al. Thromb Res. 2009 Jan. 6).

The invention also relates to the use of free Protein S having APC cofactor activity and of total Protein S as indicators of autoimmune protein S deficiency. The invention also relates to a method for diagnosing autoimmune protein S deficiency in a subject, said method comprising determining in a blood sample obtained from said subject the level of free Protein S having Activated Protein C (APC) cofactor activity and the level of total Protein S.

The methods of the invention may be used in combination with traditional methods used to diagnose thrombophilia or a systemic auto-immune disease in a subject. Typically a physician may also consider other clinical or pathological parameters used in existing methods to diagnose thrombophilia or a systemic auto-immune disease. Thus, results obtained using methods of the present invention may be compared to and/or combined with results from other tests, assays or procedures performed for the diagnosis of thrombophilia or a systemic auto-immune disease. Such comparison and/or combination may help provide a more refine diagnosis.

Determining in a blood sample the level of free Protein S having APC cofactor activity and the level of total Protein S may be performed by any known method in the art. The levels may be measured by using standard immunodiagnostic techniques, including immunoassays such as competition, direct reaction, array chips, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, gas chromatography, high performance liquid chromatography (HPLC), size exclusion chromatography, solid-phase affinity, etc.

For example the level of free Protein S having APC cofactor activity may be determined by immunologic (such as the commercially available ASSERACHROM® free Protein S assay of Diagnostica Stago, France) and functional assays (such as the commercially available STACLOT® Protein S assay of Diagnostica Stago, France). The APC cofactor activity of PS can be measured in assays based on modified activated partial thromboplastin time (APTT) and prothrombin time (PT) formats.

The level of total Protein S may be determined by immunologic (such as the commercially available ASSERACHROM® total Protein S assay of Diagnostica Stago, France).

In a particular embodiment, the level of free Protein S having APC cofactor activity may be determined with a binding partner capable of selectively interacting with free Protein S having APC cofactor activity. The binding partner may bind to the specific amino acids of protein S which are responsible for recognition of activated protein C. Typically the binding partner may bind to the gamma-carboxyglutamic acid domain of the protein S. This domain is involved in the Protein S interaction with APC (Saller et al. Blood. 2005 Jan. 1; 105(1):122-30).

For example, the binding partner may be an anti-protein S antibody that may be polyclonal or monoclonal or a fragment or a derivative thereof. In another example, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g. U.S. Pat. No. 4,946,778) can be adapted to produce single chain antibodies. Antibodies useful in practicing the present invention also include fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The aforementioned assays may involve the binding of the binding partner (ie. Antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The binding partners of the invention such as antibodies or aptamers may be labelled with a detectable molecule or substance, such as a fluorescent molecule, a radioactive molecule or any others labels known in the art. Labels are known in the art that generally provide (either directly or indirectly) a signal.

As used herein, the term “labeled”, with regard to the antibody or aptamer, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

In a particular embodiment, an ELISA method may be suitable for determining in a blood sample the level of free Protein S having APC cofactor activity and the level of total Protein S, wherein the wells of a microtiter plate are coated with a set of antibodies against free Protein S having APC cofactor activity and with a set of antibodies able to detect total Protein S. A blood sample is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

In another particular embodiment, determining in a blood sample the level of free Protein S having APC cofactor activity and the level of total Protein S may be performed with an array chip. Such an array technology allows a large number of experiments to be performed simultaneously on a single substrate, commonly known as a biochip when used for biological analytes. Example of array chips are described in the international patent document WO2007012885 and Dupuy A M. et al. (2005), Weinberger S R et al. (2000) and Jain K K et al. (2000).

For example the binding partner for free Protein S having APC cofactor activity and for total Protein S may be immobilized at the surface of said array chip. The blood sample obtained from said subject is then deposited in the array chip. After a period of incubation sufficient to allow the formation of complexes, the array chip is then washed to remove unbound moieties. In a second step, determining the level of free Protein S having APC cofactor activity and the level of total Protein S with a second binding partner specific for said free Protein S having APC cofactor activity and said total Protein S. In a preferred embodiment, said binding partner is labelled thus allowing the formation of a set of “spots” (coloured deposit) specific for the free Protein S or for the total Protein S. For example, detection and quantification may be performed by analysing the spots in said array chip with a specific detector.

Yet another object of the invention relates to the invention relates to a kit comprising:

a) means for detecting free Protein S having APC cofactor activity; and
b) means for detecting total Protein S.

Typically said kit comprises:

a) a binding partner which selectively interacts with free Protein S having APC cofactor activity; and
b) a binding partner able to detect total Protein S.

Typically said binding may be an antibody that may be polyclonal or monoclonal or a fragment or a derivative thereof.

Typically the antibodies may be labelled as above described.

The kit may also contain other suitably packaged reagents and materials needed for the particular detection protocol, including solid-phase matrices, if applicable, and standards.

In a preferred embodiment, the methods according to the present invention are carried out by determining the level of free Protein S having Activated Protein C

(APC) cofactor activity. Alternatively, the same methods may be carried out by determining the level of free Protein S.

The invention will further be illustrated in view of the following figures and examples.

### FIGURES LEGEND

FIG. 1. Ratio of “protein S activity” versus “total protein S”.

Levels of protein S activity and total protein S were measured in the plasma of HIV-positive patients with (full circles) and without (empty circles) nodular regenerative hyperplasia, from HIV-negative patients with nodular regenerative hyperplasia (full squares) and from healthy controls (empty squares). The figure depicts for each patient the ratio of “protein S activity” versus “total protein S”. Statistical significance was assessed using the Student T test.

FIG. 2. Levels of protein S-specific IgG in HIV-positive patients with nodular regenerative hyperplasia.

A. Plasma from HIV-positive patients with (grey circles) and without (empty circles) nodular regenerative hyperplasia, from HIV-negative patients with nodular regenerative hyperplasia (grey and black squares) and from healthy controls (empty squares) were incubated in protein S-coated ELISA plates. Bound IgG were detected using peroxidase-coupled polyclonal anti-human IgG and its substrate. The depicted binding intensities were measured at plasma dilutions of 1/50 as the optical density (OD) scored at 492 nm. Recognition of coated protein S was dependent on the dose of IgG (not shown). B. Inhibitory activity of IgG purified from the plasma of HIV-infected patients with nodular regenerative hyperplasia. IgG (0 to 2 milligrams per milliliter) purified from the plasma of the patients was incubated with recombinant protein S (20 micrograms per milliliter). The protein C co-factor activity of protein S was then measured in a functional coagulation assay as explained. The specific inhibitory activity of IgG is expressed in units per milligram, and represents the inverse of the IgG concentration that yields 50 percent of protein S inhibition. Bars represent medians. P values are indicated on the graph. Data are from at least two independent experiments. In the case of HIV-negative patients, the two patients with systemic lupus erythematosus are depicted as black squares.

FIG. 3. Correlation between the ratio of “protein S activity” versus “total protein S”, and the IgG-mediated inhibition of Protein S.

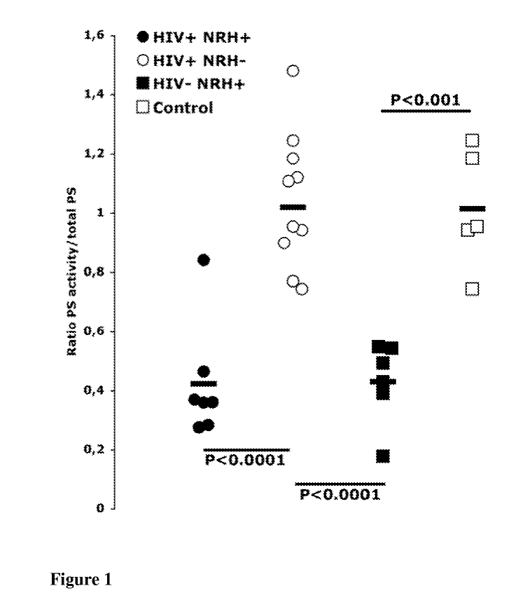

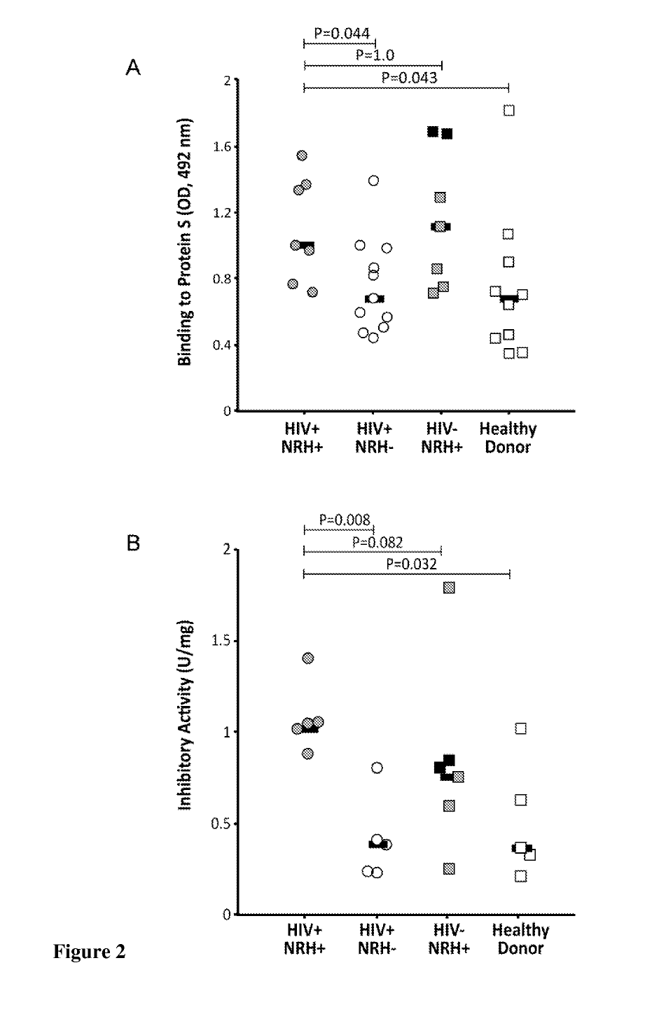

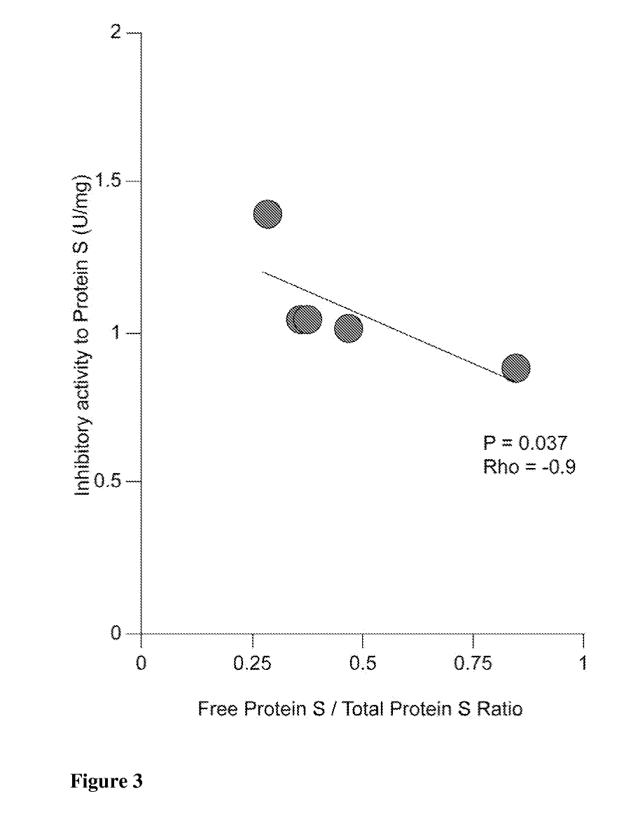

The inhibitory activity of IgG purified from the plasma of 5 HIV-infected patients with nodular regenerative hyperplasia (see FIG. 2B) was plotted as a function of the ratio of free Protein S versus Total Protein S (see FIG. 1). The significance of the correlation was assessed using the non-parametric Spearman correlation test (Rho=−0.9; P=0.037).

TABLE 1
Haemostatic assessment in cases and controls

| | HIV-positive patients | | HIV-positive patients | | HIV-negative patients | | | | |
|---|---|---|---|---|---|---|---|---|---|
| | with nodular | | without nodular | | with nodular | | Anonymous | | |
| | regenerative | | regenerative | | regenerative | | blood donors | | |
| | hyperplasia (n = 13) | | hyperplasia (n = 16) | | hyperplasia (n = 8) | | (n = 10) | | |
| Factor II level—percent | 94 | (85-108) | 104 | (99-116) | 100 | (92-106) | NA | | P = 0.08† |
| of normal | | | | | | | | | |
| Protein S activity—percent | 52 | (46-56) | 84 | (70-99) | 80 | (76-88) | NA | | P < 0.001† |
| of normal | | | | | | | | | |
| Total Protein S percent | 117 | (95-144) | 102 | (99-118) | 169 | (154-226) | 101 | (93-106) | P < 0.005† |
| of normal | | | | | | | | | |
| Ratio Protein S | 0.36 | (0.32-0.42) | 0.95 | (0.83-1.15) | 0.46 | (0.40-0.53) | 0.95 | (0.94-1.18) | P < 0.001† |
| activity vs total | | | | | | | | | |
| Protein S | 136 | (29-238) | 158 | (136-175) | 506 | (388-813) | NA | | P < 0.001† |
| C4bBP—percent of | | | | | | | | | |
| normal | | | | | | | | | |
| IgG level—mg/ml | 19.9 | (17.5-23.7) | 10.0 | (7.2-11.9) | 13.9 | (8.7-17.9) | 8.9 | (8.5-10.3) | P = 0.002† |
| IgG inhibitory activity | 1.05 | (1.02-1.05) | 0.38 | (0.24-0.41) | 0.78 | (0.63-0.83) | 0.37 | (0.33-0.63) | P = 0.022† |
| towards protein S— | | | | | | | | | |
| arbitrary units | | | | | | | | | |

*Values are presented as medians and interquartile ranges or otherwise as indicated. C4bBP stands for C4b binding protein, NA not available.
†P values computed for multiple comparisons

TABLE 2
Haemostatic assessment in patients with systemic
lupus erythematosus and healthy donors

| | Healthy donors* | | SLE patients* | | P† |
|---|---|---|---|---|---|
| Number | 8 | | 39 | | |
| C4BbP | 112 | (91-146) | 143 | (77-227) | 0.03 |
| Total Protein S | 83 | (43-151) | 129 | (23-700) | 0.09 |
| Free Protein S | 88 | (61-94) | 75 | (17-130) | 0.40 |
| Free protein S/ | 0.99 | (0.61-1.5) | 0.52 | (0.16-1.14) | 0.001 |
| total protein S Ratio | | | | | |

*Values are indicated in % (range), where 100% is the value in normal plasma
†Differences validated using the non parametric Mann-Whitney test

### EXAMPLE 1

### Abstract

We compared 13 consecutive HIV-positive patients with unexplained nodular regenerative hyperplasia to 16 consecutive HIV-positive patients without nodular regenerative hyperplasia, to eight HIV-negative patients with nodular regenerative hyperplasia from an identified cause and to 10 anonymous healthy blood donors. Patients and controls were screened for deficiency protein S activity and anti-protein S IgG antibodies. The anti-protein S activity of purified IgG from patients and controls was assessed in a functional test of activation of protein C in which protein S serves as a cofactor. A full liver CT-portography was realized on the liver explant of a case patient.

The CT-portography disclosed diffuse obliterative portal venopathy. Levels of protein S activity were lower among patients with HIV-associated nodular regenerative hyperplasia when compared to HIV-positive patients without nodular regenerative hyperplasia and to HIV-negative patients with nodular regenerative hyperplasia (P<0.005 for all comparisons). HIV-positive patients with nodular regenerative hyperplasia had significantly higher levels of anti-protein S IgG than HIV-negative patients with nodular regenerative hyperplasia and healthy controls. Purified IgG from patients with HIV-associated nodular regenerative hyperplasia specifically inhibited the protein S-dependant protein C activation.

Conclusion: Acquired autoimmune protein S paucity and secondary thrombophilia appear to be causes of obliterative portal venopathy and compensatory nodular regenerative hyperplasia in HIV-positive patients.

### Methods

### Study Design

16 HIV-infected patients with biopsy-proven nodular regenerative hyperplasia of the liver were referred to the liver unit of Cochin University Hospital (Paris, France). Three of these 16 patients were co-infected with the hepatitis C virus and were excluded from the present analysis. All common causes of chronic liver disease were ruled out in the other 13 patients. Genome amplifications of viral hepatitis B and C were negative, excluding overt or occult hepatitis B and/or hepatitis C infection(s).

Hepatitis B core (HBc) antibodies (IgG) were positive in 6/13 patients. There was no past or recent history of excessive alcohol consumption (>20 g/day), ferritin and transferrin saturation blood levels were normal, anti-liver auto-antibodies and anti-nuclear antibodies were tested negative. Serum alpha-1-antitrypsin, copper and ceruloplasmin levels were normal—ruling out hemochromatosis, auto-immune hepatitis, alpha-1-antitrypsin deficiency and Wilson's disease, respectively. None of the patients had significant co-morbidity, especially concerning cardiac, hematological or renal pathologies. There was no history of toxic exposure (vitamin A, copper sulphate, vinyl chloride monomer, thorium sulphate, Spanish toxic oil, or arsenic salts), no past or ongoing hormonal therapy or herbal medicine treatment.

We compared the levels of protein S activity of cases patients, of 16 consecutive HIV-positive patients with strictly normal liver function tests and of eight patients with nodular regenerative hyperplasia secondary to an identified cause. The eight HIV-negative patients with nodular regenerative hyperplasia presented with heterogeneous underlying diseases that included five kidney transplants, two lupus, one bartonellosis. On the basis of sample availability, we used the serum of either seven or five HIV-positive patients with nodular regenerative hyperplasia, for testing the recognition of protein S in ELISA and the inhibitory activity of purified IgG towards Protein S, respectively. The clinical profiles for these patients were similar to that of the remaining patients, especially concerning CD4 cell count, and there was no bias in the groups studied (P=0.127 for the comparison). Ten samples of blood from anonymous healthy blood donors that served as controls for the IgG assays. Informed consent was obtained from all patients, and the institutional review board of our hospital approved the study.

### Prothrombotic Haemostatic Defect Assessment

All patients were screened for lupus anticoagulant, antiphospholipid antibodies of IgG and IgM isotypes, antithrombin and protein C functional deficiencies, protein S functional deficiency and G1691A factor V and G202110A factor II mutations. Levels of total protein S and of free Protein S were measured using commercially available ELISA (ASSERACHROM® total Protein S assay and ASSERACHROM® free Protein S assay of Diagnostica Stago, France), as indicated by the manufacturer.

Measurement of C4b-Binding Protein in Plasma

Levels of C4b-binding protein in the plasma from patients and controls were assessed using the commercially available Liatest® C4b-binding protein chromogenic assay as instructed (Diagnostica Stago, France).

### Quantification of Anti-Protein S IgG by Enzyme-Linked Immunosorbent Assay (ELISA)

ELISA plates were coated with recombinant human protein S in phosphate buffer saline (PBS) at two micrograms per milliliter and left overnight at four degrees Celsius (Morboeuf et al. Thromb Res 2000, 100:81-88). The plates were washed with PBS containing 0.2 percent Tween 20 and blocked with PBS with one percent Bovine Serum Albumin, then left for one hour at room temperature. Plasma samples from patients and controls including healthy blood donors were incubated in serial dilutions for two hours at room temperature. After extensive washing of the wells, bound IgG was revealed using polyclonal goat anti-human IgG antibodies coupled to peroxidase (Clone JDC-10, Southern biotechnology) and its substrate. The chromogenic product was read at 492 nanometers using a Genyos (TECAN).

Purification of IgG from Plasma

IgG was purified from the plasma of patients and healthy blood donors by affinity-chromatography on protein G-Sepharose (Amersham Pharmacia Biotech, Buckinghamshire, England). Plasma was incubated with protein G-Sepharose in PBS with 0.01 percent azide (PBS/azide), left overnight at four degrees Celsius. After extensive washing with PBS/azide, IgG was eluted using 0.2 mol per liter glycine-HCl (pH 2.8) and neutralized using three molar Tris. IgG was dialyzed against PBS for four hours at four degrees Celsius and quantified by spectrometry at 280 nanometers.

### Functional Assay for Inhibition of Protein S

Recombinant human protein S (20 micrograms per milliliter) was incubated in Owren-Koller buffer alone or with purified IgG (0.5, 1 and 2 milligrams per milliliter) for two hours at 37 degrees Celsius (Morboeuf et al. Thromb Res 2000, 100:81-88). Samples were then added to a mixture of human plasma depleted in protein S, human activated protein C and bovine activated factor V. Coagulation was initiated with calcium chloride (0.025 mol per liter) and the time taken to coagulate was measured using a coagulometer KC10 micro (Amelung, Lemgo, Germany) and compared to a standard curve obtained with serial dilutions of recombinant protein S (30 to 0.33 micrograms per milliliter). We calculated the specific inhibitory activity of IgG expressed in units per milligram that represents the inverse of the IgG concentration that yields 50 percent of protein S inhibition.

### Statistical Analysis

Continuous variables are presented as median and interquartile range or means and standard errors of the mean. Categorical variables are presented as counts and percentages. The differences between groups were assessed with the Mann-Whitney U test and the Kruskal-Wallis H tests. All P values are two-sided and the type I error was set to 5 percent. All statistical analyses were performed using SPSS software, version 16 (SPSS Inc, Chicago, Ill., USA).

### Results

### Clinical Characteristics

The median age of patients with HIV-associated nodular regenerative hyperplasia was 41 years. There were nine men and four women. All had a long-lasting HIV-infection (median length of known HIV-positivity of 12 years) with various routes of contamination. All were considered adequately immune-restored by highly active antiretroviral therapy with a median CD4 T cell count at the time of diagnosis of 265 cells per microliter and a normal proportion of CD4 cells. All were or had been exposed to didanosine (among various antiretroviral treatments).

The initial mode of presentation was unexplained abnormal liver-function tests (elevation of alkaline phosphatase with mild thrombopenia) in more than half of the patients. The remainder presented with direct or indirect signs of portal hypertension.

The median delay between the first noted liver abnormality and diagnosis of nodular regenerative hyperplasia was 17 months.

Eleven patients (85 percent) had esophageal varices and hypertensive gastropathy on upper endoscopy. Six of them experienced gastrointestinal hemorrhage at the time of diagnosis or during follow-up. One patient developed chronic diarrhea and severe denutrition related to portal hypertensive exudative enteropathy. Four patients underwent liver transplantation for liver insufficiency and portal hypertension. The median delay between the diagnosis of nodular regenerative hyperplasia and liver transplantation was 37 months (range 24 to 47). Two patients had portal vein thrombosis at the time of the liver transplantation.

### Pathology and Radiology

Access to the explant of the last transplanted patient provided a glimpse of the mechanism of HIV-associated nodular regenerative hyperplasia at both radiological and pathological levels. A CT-portography of the whole liver revealed figures of obliterative portal venopathy with a diffuse occlusion of the distal intrahepatic portal branches. The pathological examination of the explant disclosed a typical aspect of obliterative portal venopathy with figures of nodular regenerative hyperplasia, sinusoidal dilatation, and hepatoportal sclerosis in the liver parenchyma. No significant fibrosis was seen.

### Prothrombotic Haemostatic Defect Assessment

Obliterative portal venopathy is frequently associated with prothrombotic disorders. We therefore screened every patient with HIV-associated nodular regenerative hyperplasia for a prothrombotic haemostatic defect. All patients with HIV-associated nodular regenerative hyperplasia had decreased levels of protein S activity (Table 1).

When compared to matched controls, the levels of protein S activity were lower in HIV-positive patients with nodular regenerative hyperplasia as compared to HIV-positive patients without nodular regenerative hyperplasia and to HIV-negative patients with nodular regenerative hyperplasia (Table 1). In contrast, the levels of total protein S were similar in the case of HIV-positive patients with and without nodular regenerative hyperplasia, and were similar to that of healthy donors (Table 1).

We then calculated, for each patient included in the study, the ratio of “protein S activity” versus “total protein S” (Table 1). HIV-positive patients with nodular regenerative hyperplasia had ratios significantly lower than HIV-positive patients without nodular regenerative hyperplasia and healthy controls (FIG. 1, P<0.001). The same was true of HIV-negative patients with nodular regenerative hyperplasia.

Levels of C4b-Binding Protein

To determine whether the decrease in free protein S could be attributed to a shift towards the form complex to the C4b-binding protein, levels of C4b-binding protein in the plasma of the patients were compared to those in the plasma of controls. HIV-infected patients with and without nodular regenerative hyperplasia presented with identical levels of C4b-binding protein in plasma (P=0.530). Unexpectedly, plasma from HIV-negative patients with nodular regenerative hyperplasia had 3.6 to 5.4-fold higher levels of plasma C4b-binding protein (Table 1), possibly explaining the significant increase in total protein S in these patients.

### Anti-protein S IgG

We investigated whether the reduced protein S levels in HIV-infected patients is due to an acquired anti-protein S humoral immune response. Interestingly, protein 5-specific IgG was detected in the plasma of healthy donors. HIV-infected patients without nodular regenerative hyperplasia showed anti-protein S IgG levels similar to those of healthy individuals. In contrast, both HIV-negative and HIV-positive patients with nodular regenerative hyperplasia had higher levels of anti-protein S IgG than those of healthy donors and than those of patients with HIV-infection without nodular regenerative hyperplasia (FIG. 2A). In the case of HIV-positive patients with nodular regenerative hyperplasia, the elevated protein S recognition by plasma IgG was associated with higher amounts of circulating IgG as compared to HIV-positive patients without nodular regenerative hyperplasia and to healthy blood donors (Table 1). Of note, patients with the highest levels of anti-protein S IgG had lupus as an underlying disease.

### Inhibitory Activity of Purified IgG Towards Protein S

We then investigated whether the anti-protein S IgG in patients with nodular regenerative hyperplasia can neutralize protein S function. Basal inhibition of protein S activity by IgG from healthy donors and HIV-infected patients without nodular regenerative hyperplasia was observed, probably owing to the limitations of our inhibitory assay. The inhibition of protein S activity was dependant on the concentration of IgG. IgG from HIV-positive patients with nodular regenerative hyperplasia showed consistently and significantly higher inhibitory activity towards protein S than IgG from healthy donors and IgG from HIV-positive patients without nodular regenerative hyperplasia. IgG from HIV-negative patients with nodular regenerative hyperplasia were heterogeneous in terms of protein S inhibition, and did not significantly differ from the inhibition observed with that of IgG from healthy donors and HIV-infected patients without nodular regenerative hyperplasia (P=0.329 and P=0.126 respectively) (Table 1, FIG. 2B). There was a significant correlation between the inhibition of protein S activity by the IgG purified from the plasma of 5 HIV-positive patients with nodular regenerative hyperplasia, and the free vs total protein S ratios (FIG. 3).

### Discussion

We present here a case-series of 13 patients with nodular regenerative hyperplasia where the only identified etiologic factor is HIV-infection. The mode of presentation was very stereotypical: long-lasting HIV-infection, exposition to antiretroviral drugs, especially didanosine, adequate immune restoration and unexplained abnormal liver-function tests or portal hypertension. Complications related to portal hypertension occurred in about half of them, and four required liver-transplantation. The mechanism of HIV-associated nodular regenerative hyperplasia is unknown to date, but does not seem to be related to HIV-infection and acquired immunodepression per se since the majority of patients were adequately immune-restored.

The CT-portography of the explant of the last transplanted patient shows that the primary lesion in HIV-associated nodular regenerative hyperplasia is diffuse obliterative portal venopathy. The obliteration of the small portal veins results in ischemia of the supplied acini and regenerative hyperplasia of the remainders in order to maintain liver cell mass. This observation is in line with seminal autopsic studies on nodular regenerative hyperplasia suggesting that the primary lesion in nodular regenerative hyperplasia is obliterative portal venopathy. We propose the term of HIV-associated obliterative portopathy (HIV-OP) to describe the present syndrome in HIV-infected patients.

Nodular regenerative hyperplasia and obliterative portal venopathy have been reported to occur in association with other systemic diseases, including rheumatic, vascular and myeloproliferative disorders, but also with certain drugs and congenital or acquired prothrombotic states, including acquired protein S deficiency.

Protein S activity was significantly lower in patients with HIV-OP as compared to matched HIV-positive controls without nodular regenerative hyperplasia and in HIV-negative patients with nodular regenerative hyperplasia of another origin. Our data thus indicate a link between protein S deficiency and the development of HIV-OP.

Interestingly, despite a marked decrease in protein S activity, the levels of total protein S in HIV-positive patients with nodular regenerative hyperplasia were identical to that of HIV-positive patients without nodular regenerative hyperplasia and healthy donors, indicating functional inactivation of circulating protein S. We indeed document increased levels of anti-protein S IgG in the plasma from patients with HIV-OP as compared with patients from the other groups, including healthy donors. Of particular interest, calculation of the ratios of “protein S activity” versus “total protein S” allowed to clearly distinguish patients with nodular regenerative hyperplasia from other patients and from healthy donors.

Anti-protein S IgG were able to inhibit the co-factor activity of protein S towards activated protein C in a functional assay. The observed significant correlation between the inhibition of protein S activity by IgG purified from HIV-positive patients with nodular regenerative hyperplasia, and the free vs total protein S ratios, suggests that reduced free vs total protein S ratios indirectly reflect the presence of inhibitory anti-protein S IgG in the patients.

Importantly, levels of C4b-binding protein were not altered in patients with HIV-OP. While levels of free protein S may be affected by multiple disease process, including liver disease and thrombosis, our data suggest that acquired anti-protein S IgG participate to the pathogenesis of nodular regenerative hyperplasia in the liver of HIV-positive patients. The presence of high levels of inhibitory anti-protein S IgG in HIV-infected patients may be explained by a persisting abnormality of B-cell activation or B-cell repertoire despite antiretroviral therapy, leading to chronic polyclonal activation (Redgrave et al. HIV Med 2005, 6:307-312). Indeed, the presence of anti-protein S antibodies was well correlated to IgG levels. The HIV-infected patients studied herein had correct CD4-positive lymphocyte cell count, and half of them had dramatically increased their CD4 cell count from a very low nadir. This is known to favor the occurrence of various manifestations of the inflammatory immune restoration syndrome, including inflammatory and bona fide, autoantibody-mediated autoimmune conditions.

Although the incidence of HIV-OP is unknown, it is likely to be more common than generally thought. Although we report here only patients with isolated HIV-OP, we have observed identical cases in patients co-infected with hepatitis C virus (Mallet et al. Gastroenterol Clin Biol 2007, 31:878-880). Because patients with HIV-OP typically present clinical features resembling those of cirrhosis, liver biopsy is indicated in most cases to confirm the diagnosis. Nevertheless, in the absence of any underlying liver disease, the diagnosis of HIV-OP should be considered in the presence of unexplained abnormal liver-function tests in an HIV-infected patient, especially if decreased level of protein S is found. In this case, patients should be screened for occult portal hypertension.

In conclusion, HIV-OP appears as a complication of treated HIV-infection under highly active antiretroviral therapy secondary to acquired autoimmune protein S deficiency.

### EXAMPLE 2

In order to investigate whether altered free/total protein S ratios may also be observed in patients with autoimmune disorders, we collected the plasma from 39 patients with systemic lupus erythematosus (SLE) from Hôpital Cochin (Paris, France). As controls, we also collected plasma from 8 healthy blood donors. Levels of total protein S and of free Protein S were measured in plasma using commercially available ELISA (ASSERACHROM® total Protein S assay and ASSERACHROM® free Protein S assay of Diagnostica Stago, France), as described above (Example 1). Levels of C4b-binding protein in the plasma from patients and controls were assessed using the commercially available Liatest® C4b-binding protein chromogenic assay (Diagnostica Stago, France).

Levels of free and total protein S did not differ significantly between patients with SLE and healthy donors (Table 2). Levels of C4b-binding protein were 1.3-fold higher in the plasma of SLE patients than in that of healthy donors (P=0.03). We then calculated the ratio of free protein S versus total protein S. The ratio was 2-fold lower for SLE patients as compared to healthy controls (Table 2, P=0.001). Interestingly, 24 out of the 39 patients included in the study (62%) had a free protein S versus total protein S ratio that was lower than the minimal value measured in the case of healthy donors (i.e., 0.61).

In conclusion, our study shows that 62% of patients with SLE demonstrate a reduced ratio of free versus total protein S.

### REFERENCES

Throughout this application, various references describe the state of the art to which this invention pertains. The disclosures of these references are hereby incorporated by reference into the present disclosure.

1. A method for diagnosing thrombophilia in a subject suffering from HIV or from a systemic auto-immune disease, said method comprising determining in a blood sample obtained from said subject the level of free Protein S having Activated Protein C (APC) cofactor activity and the level of total Protein S.

2. A method for diagnosing autoimmune protein S deficiency in a subject, said method comprising determining in a blood sample obtained from said subject the level of free Protein S having Activated Protein C (APC) cofactor activity and the level of total Protein S.

3. A method according to claim 1, comprising the step of calculating the ratio free Protein S having APC cofactor activity/total Protein S, wherein a low ratio is indicative of the presence of anti-protein S autoantibodies that neutralize the cofactor activity of the free protein S.

4. A method according to claim 1, wherein said subject is suffering from HIV.

5. A method according to claim 1, wherein said subject is suffering from a systemic auto-immune disease.

6. A method according to claim 5, wherein said systemic auto-immune disease is systemic lupus erythematosus (SLE), the antiphospholip syndrome, Behcet's disease, Common variable immune deficiency (CVID), viral-induced prothrombotic states, thrombotic thrombocytopenic purpura.

7. A kit comprising:
a) means for detecting free Protein S having APC cofactor activity; and
b) means for detecting total Protein S.

8. A kit according to claim 7, wherein said kit comprises:
a) a binding partner which selectively interacts with free Protein S having APC cofactor activity; and
b) a binding partner able to detect total Protein S.

9. (canceled)

10. A method according to claim 2, comprising the step of calculating the ratio free Protein S having APC cofactor activity/total Protein S, wherein a low ratio is indicative of the presence of anti-protein S autoantibodies that neutralize the cofactor activity of the free protein S.

11. A method according to claim 2, wherein said subject is suffering from HIV.

12. A method according to claim 3, wherein said subject is suffering from HIV.

13. A method according to claim 2, wherein said subject is suffering from a systemic auto-immune disease.

14. A method according to claim 3, wherein said subject is suffering from a systemic auto-immune disease.

15. A method according to claim 4, wherein said subject is suffering from a systemic auto-immune disease.

16. A method according to claim 13, wherein said systemic auto-immune disease is systemic lupus erythematosus (SLE), the antiphospholip syndrome, Behcet's disease, Common variable immune deficiency (CVID), viral-induced prothrombotic states, thrombotic thrombocytopenic purpura.

17. A method according to claim 14, wherein said systemic auto-immune disease is systemic lupus erythematosus (SLE), the antiphospholip syndrome, Behcet's disease, Common variable immune deficiency (CVID), viral-induced prothrombotic states, thrombotic thrombocytopenic purpura.

18. A method according to claim 15, wherein said systemic auto-immune disease is systemic lupus erythematosus (SLE), the antiphospholip syndrome, Behcet's disease, Common variable immune deficiency (CVID), viral-induced prothrombotic states, thrombotic thrombocytopenic purpura.
